(19) United States
(12) Patent Application Publication
(10) Pub. No.: US 2014/0336637 A1
(43) Pub. Date: Nov. 13, 2014

(54) SYSTEMS AND METHODS FOR TEMPERATURE MONITORING AND CONTROL DURING AN ABLATION PROCEDURE

(71) Applicant: BOSTON SCIENTIFIC SCIMED, INC., Maple Grove, MN (US)

(72) Inventors: SUMIT AGRAWAL, WOODBURY, MN (US); PRAMODSINGH HIRASINGH THAKUR, WOODBURY, MN (US); DAVID RAAB, MINNEAPOLIS, MN (US); STEVE HONGXI ZHANG, BLAINE, MN (US); TRAVIS J. SCHAUER, DELANO, MN (US)

(73) Assignee: BOSTON SCIENTIFIC SCIMED, INC., Maple Grove, MN (US)

(21) Appl. No.: 14/273,248

(22) Filed: May 8, 2014

Related U.S. Application Data
(60) Provisional application No. 61/821,142, filed on May 8, 2013.

(51) Int. Cl. A61B 18/14

(52) U.S. Cl. CPC A61B 18/1492; A61B 2018/00797
USPC 606/41

(57) ABSTRACT

A medical system configured for nerve modulation can include an elongate shaft, having a distal end region and a proximal end region is disclosed. Adjacent the distal end region an ablation electrode can be disposed. The system can further include a first optical fiber, having a proximal end and a distal end, extending along an outer surface of the elongate shaft, and in turn a number of (fiber Bragg Grating) FBG sensors therein. The FBG sensors can be positioned adjacent to the ablation electrode. An optical read out mechanism can be optically coupled to the optical fiber to transmit light into the optical fiber and detect light reflected from the FBG sensor. Here, the detected light, reflected from FBG temperature sensors, encodes local temperatures at each of the FBG temperature sensors.

### CROSS-REFERENCE TO RELATED APPLICATIONS

This application claims priority under 35 U.S.C. §119 to U.S. Provisional Application Ser. No. 61/821,142, filed May 8, 2013, the entirety of which is incorporated herein by reference.

### TECHNICAL FIELD

The present disclosure relates generally to devices and methods for monitoring and controlling temperature within a body lumen. More particularly, the disclosure relates to monitoring and controlling temperature within a body lumen during a renal nerve modulation or other procedure such as during use of cardiac electrophysiology catheters.

### BACKGROUND

Certain treatments require the temporary or permanent interruption or modification of select nerve function or cardiac tissue. One example treatment is renal nerve ablation, which is sometimes used to treat conditions related to congestive heart failure or hypertension. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves, including renal nerves, run along the walls of or in close proximity to blood vessels and thus can be accessed via the blood vessels. In some instances, it may be desirable to ablate perivascular renal nerves using radiofrequency energy. The target nerves must be heated sufficiently to make them nonfunctional, however tissue adjacent to the nerves may also be damaged. It may be desirable to provide for alternative systems and methods for intravascular nerve modulation that reduce damage to surrounding tissues.

### SUMMARY

This disclosure is directed to several alternative designs, materials, and methods of manufacturing and using medical device structures and assemblies, for monitoring and/or controlling in situ or local temperature during medical procedures, such as during a renal nerve modulation procedure.

Accordingly, one illustrative embodiment discloses a medical system for modulating nerves. The medical system can include an elongate shaft having a proximal end region and a distal end region, and an ablation electrode disposed adjacent the distal end region. The medical system can also include an optical fiber having a proximal end and a distal end, extending along an outer surface of the elongate shaft. More particularly, the optical fiber can include a number of fiber Bragg Grating (FBG) temperature sensors therein, which is/are configured to be positioned adjacent to the ablation electrode. Further, an optical read-out mechanism can be optically coupled to the optical fiber. The optical read-out mechanism can be configured to transmit light into the optical fiber and detect light reflected from the FBG temperature sensor. The detected light reflected from the FBG temperature sensor encodes local temperature at the FBG temperature sensor.

Another aspect of the present disclosure describes a medical device for modulating nerves. The medical device can include an elongate shaft having a distal region, and a balloon coupled to that distal region. The balloon can have an inner conductive layer, an outer conductive layer and an intermediate non-conductive layer disposed between the inner layer and the outer layer. Further, an electrode can be disposed within the balloon, alongside a virtual electrode that includes a conductive region defined on the balloon. An optical fiber having a proximal end and a distal end can extend along an outer surface of the elongate shaft and include a number of fiber Bragg Grating (FBG) temperature sensor(s) therein. An optical read-out mechanism can be optically coupled to the optical fiber and can include a mechanism to transmit light into the optical fiber and detect light reflected from the FBG temperature sensor(s). The detected light reflected from the FBG temperature sensor encodes local temperature at the FBG temperature sensor.

Certain embodiments of the present disclosure describe a deflectable medical device. The medical device can include a catheter shaft having a distal end, and an ablation electrode disposed at that distal end. The medical device can further include a deflection mechanism coupled to the catheter shaft, where the deflection mechanism includes a deflection body and a pull wire coupled to that deflection body. Further, a flex member can be disposed adjacent the deflection mechanism. Additionally, an optical fiber having a proximal end and a distal end can extend along an outer surface of the elongate shaft, and may include a number of fiber Bragg Grating (FBG) temperature sensor(s) therein. Furthermore, an optical read-out mechanism can be optically coupled to the optical fiber, and configured to transmit light into the optical fiber and detect light reflected from the FBG temperature sensor(s). The detected light reflected from the FBG temperature sensor encodes local temperature at the FBG temperature sensor.

Although discussed with specific reference to use with the renal nerves of a patient, the medical systems and devices in accordance with the disclosure may be adapted and configured for use in other parts of the anatomy, such as the nervous system, the circulatory system, the respiratory system or other parts of the anatomy of a patient.

The above summary of an example embodiment is not intended to describe each disclosed embodiment or every implementation of the present disclosure.

### BRIEF DESCRIPTION OF THE DRAWINGS

The disclosed subject matter may be more completely understood in consideration of the following detailed description of various embodiments in connection with the accompanying drawings, in which:

FIG. 1 is a schematic view of an illustrative medical system in situ, configured for monitoring an intravascular temperature according to aspects of the present disclosure.

FIG. 2 is a schematic illustration of elements of an illustrative optical fiber-based sensor system.

FIG. 3A is an illustrative balloon catheter device according to aspects of the present disclosure.

FIG. 3B is an illustrative cross-sectional view of the balloon catheter device of FIG. 3A.

FIG. 4 is a side view of the balloon catheter device of FIG. 3A.

FIG. 5 is a side view of another illustrative embodiment of a balloon catheter device.

FIG. 6 is an illustrative deflectable medical device according to aspects of the present disclosure.

FIG. 7 is a side view of the deflectable medical device of FIG. 6.

While the disclosed subject matter is amenable to various modifications and alternative forms, specifics thereof have been shown by way of example in the drawings and will be described in detail. It should be understood, however, that the intention is not to limit aspects of the disclosed subject matter to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

For purposes of this disclosure, “proximal” refers to the end closer to the device operator during use, and “distal” refers to the end further from the device operator during use.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended to be exemplary. Selected features of any illustrative embodiment may be incorporated into an additional or alternative embodiment unless clearly stated to the contrary.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with one embodiment, it should be understood that such feature, structure, or characteristic may also be used in connection with other embodiments whether or not explicitly described unless clearly stated to the contrary.

While the devices and methods described herein are discussed relative to renal nerve modulation, it is contemplated that the devices and methods may be used in other treatment locations and/or applications where nerve modulation and/or other tissue modulation including heating, activation, blocking, disrupting, or ablation are desired, such as, but not limited to: blood vessels, urinary vessels, or in other tissues via trocar and cannula access. For example, the devices and methods described herein can be applied to hyperplastic tissue ablation, cardiac ablation, pulmonary vein isolation, bronchial thermoplasty, tumor ablation, benign prostatic hyperplasia therapy, nerve excitation or blocking or ablation, modulation of muscle activity, hyperthermia or other warming of tissues, etc. In some instances, it may be desirable to ablate perivascular renal nerves with radiofrequency ablation.

For a renal nerve modulation procedure, it may be important to control the temperature at the sight of ablation in the range of 50-100° C. A treatment temperature lower than an optimal temperature (based on the desired treatment) may lead to a sub-optimal treatment. Conversely a treatment temperature higher than an optimal temperature (based on the desired treatment) could potentially damage more tissue than required and possible cause dangerous side effects. The need for temperature control may be equally critical during cardiac ablation if not more. Greater temperatures carry the risk of causing “steam pops” which are essentially mini explosions within the burnt tissue that can cause chunks of tissue to dislodge causing pulmonary embolism or stroke. The tissue surface temperature may be an important determination of when steam pops occur. It may be desirable to place a temperature sensor as close to the tissue as possible so that it captures tissue temperature and thus help in predicting steam-pop zones, or other undesirable conditions.

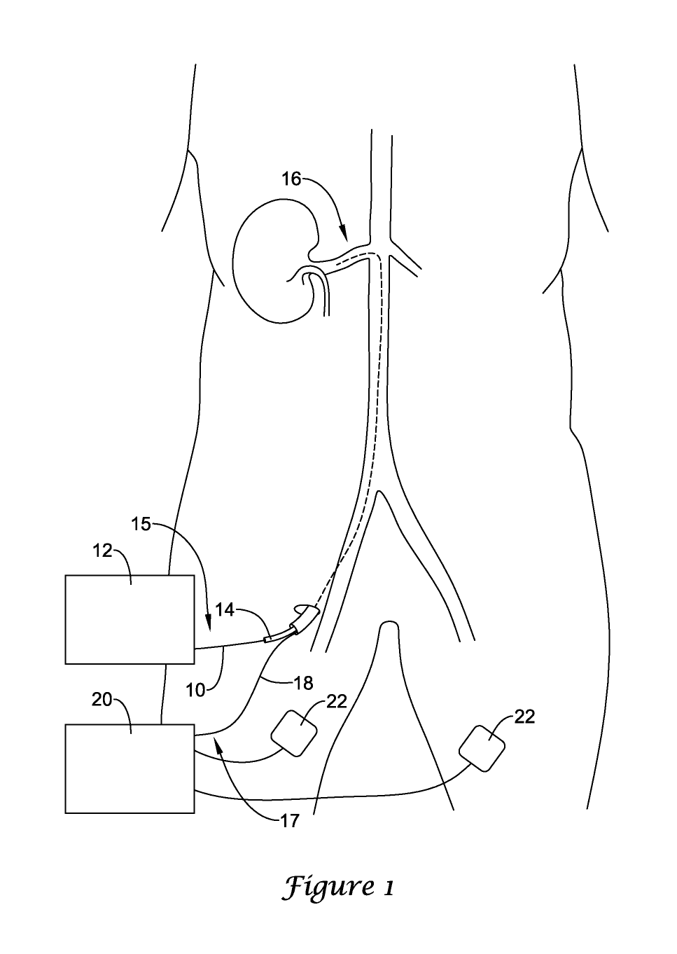

FIG. 1 is a general schematic view illustrating an exemplary temperature monitoring system configured for monitoring temperature at/adjacent a vessel wall within a patient. More particularly, FIG. 1 depicts the use of a temperature monitoring system as well as a renal nerve modulation system. In some embodiments, the temperature monitoring system and nerve modulation system may be considered parts of a single integrated system.

In further detail, the temperature monitoring system can include an optical fiber 10 that includes one or more fiber Bragg grating (FBG) temperature sensors (not shown in FIG. 1) disposed at one or more corresponding locations along a length of the optical fiber 10. For ease of reference, FBG temperature sensors may be referred to as FBG sensors hereinafter. At a proximal end region 15, the optical fiber 10 may be coupled optically to an optical read-out mechanism 12. The optical fiber 10 may be attached to and/or integrated with a support structure 14. The support structure 14 may be deployed within at least one vessel of a patient along an extent of the vessel, where it may substantially fix the optical fiber 10 within the vessel. At least one, some, or all of the FBG sensors, is/are in effective thermal contact with the vessel wall.

At a distal end region 16, the optical fiber 10 may be deployed along an extent of the vessel in a generally longitudinal or helical path, although other deployment paths are contemplated. In such cases, the support structure 14 may be deployed in the same path as the optical fiber 10, although this is not necessary. Such configurations and other embodiments are described later in the disclosure.

The support structure 14 having the optical fiber 10 couple thereto, and configured for delivery into a body vessel, may expand and/or include a deflection mechanism for deployment in the vessel. Accordingly, the temperature monitoring system may include suitable components to facilitate delivery and deployment of both the optical fiber 10 and the support structure 14 to a target location. Moreover, any suitable material may be used to form the support structure 14. In some illustrative embodiments, the support structure 14 may be fabricated from non-conducting polymers.

FIG. 1 illustrates elements of a renal nerve modulation system which may be used in concert with the temperature monitoring system, although either system may be practiced independently of the practice or presence of the other system. By way of further description, the renal nerve modulation system may include an elongate conductor 18, which may be coupled to a movable and/or deflectable ablation tip (not shown in FIG. 1) in the distal end region 16. At a proximal end region 17, the conductor 18 may be coupled to a control and power unit 20, which may supply electrical energy to the ablation electrode (not shown in FIG. 1), disposed at the distal end region 16. Return electrode patches 22 may be optionally supplied on the limbs or at another conventional location on the patient's body to complete the circuit.

In addition, control and power unit 20 may also be utilized to supply/receive the appropriate electrical energy and/or signal to activate one or more sensors disposed at or near a distal end of the renal nerve modulation system. When suitably activated, the electrodes are capable of ablating tissue as described below. The terms electrode and electrodes may be considered to be equivalent to elements capable of ablating adjacent tissue in the disclosure which follows. A proximal hub (not illustrated) having ports for a guidewire, an inflation lumen and a return lumen may also be included in the nerve modulation system.

The control and power unit 20 may include monitoring elements to monitor parameters such as power, voltage, pulse size, temperature, force, contact, pressure, impedance and/or shape and other suitable parameters, with sensors mounted along renal nerve modulation system, as well as suitable controls for performing the desired procedure. In some embodiments, the power unit 20 may control a radiofrequency (RF) electrode and, in some instances, may “power” other electrodes including so-called “virtual electrodes” described herein. The electrode may be configured to operate at a suitable frequency and generate a suitable signal. It is further contemplated that other ablation devices may be used as desired, for example, but not limited to resistance heating, ultrasound, microwave, and laser devices and these devices may require that power be supplied by the power unit 20 in a different form.

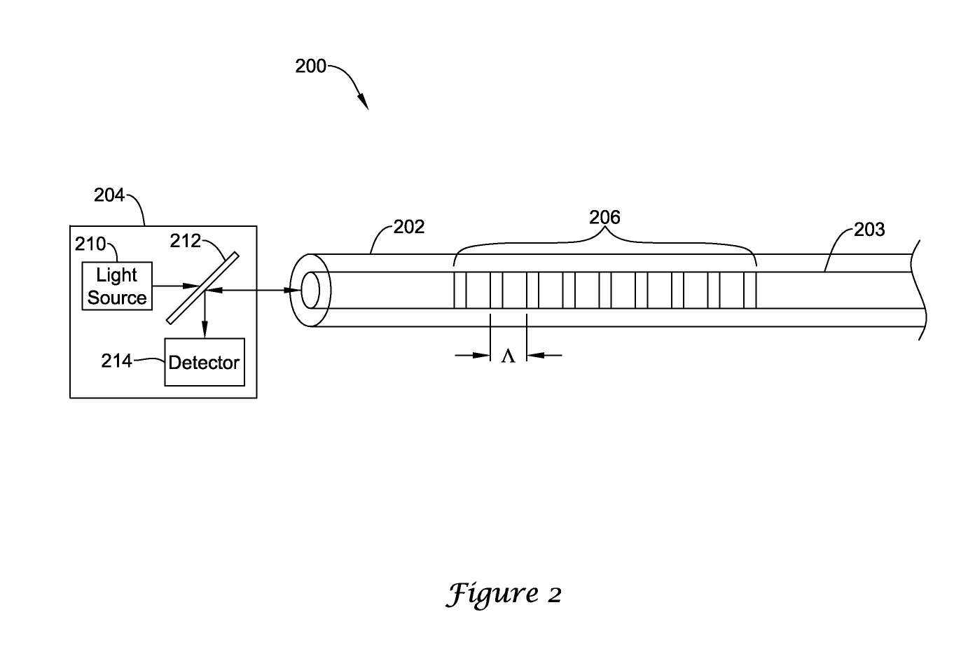

FIG. 2 is a schematic illustration of elements of an illustrative optical fiber-based sensor system 200, which may share features with the temperature monitoring system of FIG. 1. Accordingly, the system 200 includes an optical fiber 202 having a fiber core 203, which may be optically coupled to an optical read-out mechanism 204. The fiber core 203 may include one or more fiber Bragg gratings (FBGs) 206 extending along a longitudinal length at desired locations of the optical fiber 202. The length of a single period of the grating structure 206, known as grating pitch A, may be on the order of approximately 0.5 μm and the entire grating structure 206 may be a few microns. Further, FIG. 2 is a simplified, schematic illustration, and does not necessarily depict all of the technical features of an optical fiber with FBGs, as would be understood by one of ordinary skill in the art. For example, optical fiber 202 may include a core, cladding, and any other suitable layers, such as a buffer coating, protective housing, etc. Moreover, fiber Bragg gratings of the present disclosure, such as FBG 206 of optical fiber 202, may be formed by any suitable method, such as via two-beam interference, phase or photo masking, point-by-point writing by laser, and so on.

Conceptually, an FBG, such as disclosed above, generally may include variations in refractive index in the core of the fiber. The refractive index variations may form a wavelength-specific grating mirror that reflects essentially all or a portion of the light at a specific reflection wavelength, while allowing the balance of light propagating in the fiber to pass. The reflection wavelength of an FBG may shift from its nominal value due to local conditions of the optical fiber at the FBG, such as (but not necessarily limited to) temperature and strain. Temperature and/or strain may each affect the refractive index and/or grating period of the FBG, resulting in a reflection wavelength shift. This effect may be exploited to form an FBG sensor. While an FBG may generally respond (in wavelength shift) to both temperature and strain, a FBG may be packaged (e.g., housed) in order to modulate or control the physical conditions observed at the FBG. For example, an FBG may be packaged in order to decouple the FBG from bending, tension, compression, torsion, or other forces. With a nearly negligible temperature coefficient of expansion of the fiber (for a glass fiber), changes in reflection wavelength for an FBG so-packaged may be attributed primarily to a change in refractive index of the fiber caused by temperature changes. While not explicitly shown, a series of packaged or multiple FBG sensors may be provided along fiber core 203. In another sensor example, an FBG may be packaged in such a way that the packaging or housing couples changes in pressure into stress in the fiber, leading to a predictable shift in reflection wavelength. An FBG chemical sensor, for example, may include a FBG housing incorporating a chemically-sensitive substrate. In general, any physical mechanism that translates a change in a physical quantity into a change in FBG reflection wavelength may potentially be used as the basis for a FBG sensor. Other sensors may be contemplated as well.

Multiple FBG sensors 206 may be manufactured on a single optical fiber (e.g., 202) such that each FBG sensor 206 has a unique reflection wavelength. Such wavelength division multiplexing makes it possible to differentiate between reflection signals from a plurality of FBG sensors on a single optical fiber 202. To avoid ambiguity in interpreting FBG reflection signals, it may be desirable to fabricate each FBG to reflect within its own dedicated wavelength band wide enough to accommodate physically-induced reflection wavelength shifts (that encode signal information) as well as the intrinsic non-zero width of the non-shifted reflection distribution. Typically, FBG temperature sensors may be allocated an approximately 1 nm wide range, while FBG strain sensors may be allocated an approximately 5 nm wide range. Wider or narrower ranges may be employed, as appropriate.

FBG sensors 206 having unique reflection wavelengths may be formed at distinct/desired locations along optical fiber 202, such that each particular reflected wavelength may then correspond to a specific sensor location along the length of the optical fiber 202.

In some cases, shifts in reflection wavelength from multiple FBG sensors may be interpreted in combination to arrive at a physical measurement. For example, a temperature reading from a FBG temperature sensor may be used to calibrate a pressure reading from an FBG pressure sensor, which by itself may be sensitive to both temperature and pressure changes. In the present disclosure, an FBG sensor may incorporate one or more fiber Bragg gratings to achieve measurement of a physical quantity.

A device such as the optical read-out mechanism 204, optically coupled to the optical fiber 202, may be configured to transmit light into the optical fiber 202 and to detect light reflected from the one or more FBG sensors 206. The resultant detected light reflected from the one or more FBG temperature sensors 206 may encode local temperatures at each of the one or more FBG temperature sensors 206. More specifically, the optical read-out mechanism 204 (and read-out mechanism 12 of FIG. 1) may be employed to measure the wavelength reflected by a FBG temperature sensor 206 of optical fiber 202. Further, the optical read-out mechanism 204 may include any suitable light source 210, which may transmit light into the fiber core 203 via an optical coupler 212. While optical coupler 212 is illustrated schematically to suggest a partially-reflective mirror or beam-splitter, any suitable optical coupler may be employed.

In an illustrative embodiment, light propagates down the optical fiber 202 and is selectively reflected by one or more fiber Bragg gratings at their specific reflection wavelengths. The specific reflection wavelengths may encode or establish information about conditions at the FBG temperature sensor 206, such as temperature, pressure, etc. Reflected light, obtained thereafter, returns to the optical read-out mechanism 204, where optical coupler 212 may direct the reflected light to a detector 214. Detection of light reflected by FBG temperature sensors 206, including determination of reflection wavelengths, may then be interpreted by other components (not shown) of the optical read-out mechanism 204 (or external to the optical read-out mechanism) in order to arrive at the desired quantities measured by the FBG sensor(s) 206.

In further detail, FBG sensors, such as the FBG sensors 206, typically consist of optical fibers, such as the optical fiber 202, with sinusoidal gratings (not shown), each having a different refractive index inserted within its fiber core (not shown). The gratings are generally formed by focal exposure of the fiber core to an interference pattern of ultra-violet radiation. Such a grating structure causes wavelengths of certain frequencies to be reflected back when a broad spectrum light is transmitted through the optical fiber, such as through the optical read-out mechanism 204 (see FIG. 2). Moreover, the refractive index of the exposed fiber core changes where the interference pattern is the brightest to produce a periodic refractive index profile. Both the effective refractive index and the grating pitch change as a function of temperature, causing the Bragg Wavelength to change to a higher or lower frequency due to an effect, referred to as thermo optic effect, enabling the encoding of the local temperature at the tissue catheter interface.

A number of different light source 210/detector 214 combinations may be employed in the optical read-out mechanism 204. In one illustrative embodiment, a broadband continuous light source may be used in conjunction with a dispersive element that distributes various wavelength components of the reflected light to different locations on a detector array. In another illustrative embodiment, a tunable laser is swept over a range of wavelengths, and a photodetector measures the intensities of reflected light corresponding to the wavelengths provided by the laser at given sweep times. Other light source and detector combinations are contemplated, and any suitable combination of light source 210 and detector 214 may be employed in optical read-out mechanism 204. Some current technologies may be able to resolve reflected wavelength shifts in the order of about a single picometer, which may translate to temperature measurement resolution in the order of about 0.1° C. In some cases, temperature measurement resolutions in the order of about 0.03° C. may be achievable.

The optical fiber-based sensor systems of the present disclosure may be deployed in vivo in any suitable manner, and may be highly compatible with applications that relate to minimally-invasive techniques.

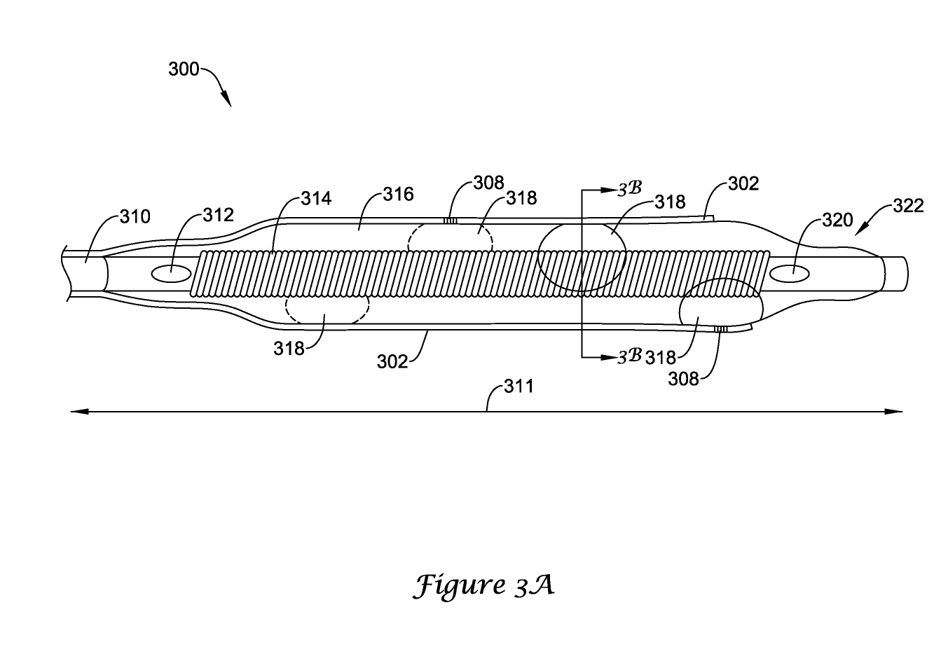

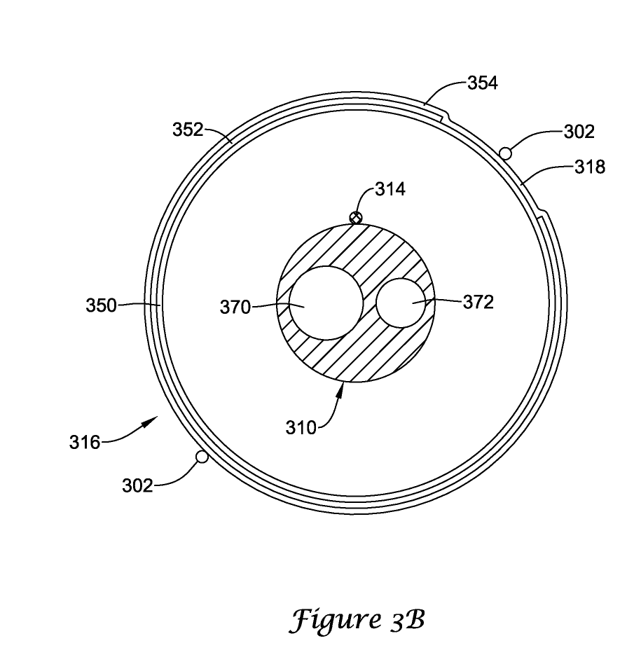

FIG. 3A illustrates a distal portion of an illustrative renal nerve modulation device 300, while the following FIG. 3B depicts an illustrative cross sectional view of the renal nerve modulation device 300 taken at line 3B-3B of FIG. 3A. Some elements of the device of FIGS. 3A and 3B may be similar to the elements described in connection with FIGS. 1 and 2. Here, it can be seen that the renal nerve modulation device 300 may include an elongate shaft or a catheter shaft 310, an expandable member or a balloon 316, coupled to the catheter shaft 310, and an ablation electrode 314. The catheter shaft 310 may extend proximally from the distal end region 322 to a proximal end region configured to remain outside of a patient's body. The ablation electrode 314 may be disposed within the balloon 316 and positioned adjacent to the distal end region 322, of the catheter shaft 310. Additional ablation electrodes 314 may also be utilized. The electrode 314 may vary and may include a number of structures such as a plurality of wires (e.g., two wires) that connect with electrode wire and, ultimately, a control and power element.

When in use, balloon 316 may be filled with a conductive fluid such as saline to allow the ablation energy (e.g., radiofrequency energy) to be transmitted from electrode 314, through the conductive fluid, to one or more virtual electrodes 318 disposed along balloon 316. While saline is one example conductive fluid, other conductive fluids may also be utilized including hypertonic solutions, contrast solution, mixtures of saline or hypertonic saline solutions with contrast solutions, and the like. The conductive fluid may be introduced through a fluid inlet 320 and evacuated through a fluid outlet 312. This may allow the fluid to be circulated within balloon 316. As described in more detail herein, virtual electrodes 318 may be generally hydrophilic portions of balloon 316. Accordingly, virtual electrodes 318 may absorb fluid (e.g., the conductive fluid) so that energy exposed to the conductive fluid can be conducted to virtual electrodes 318 such that virtual electrodes 318 are capable of ablating tissue.

A cross-sectional view of the device 300 distal to fluid outlet 312 is illustrated in FIG. 3B. A guidewire lumen 370 and a fluid inlet lumen 372 may be present as well as electrode 314. The balloon 316 may include an inner layer 350, an outer layer 354, and an intermediate layer 352, disposed between the inner layer 350 and the outer layer 354. In some embodiments, the balloon 316 may lack the outer layer 354. The virtual electrodes 318 may be formed in the balloon 316 by the absence of the intermediate layer 352. The inner and outer layers 350, 354 may include a hydrophilic, hydratable, RF permeable, and/or conductive material. One example material is hydrophilic polyurethane (e.g., TECOPHILIC® TPUs such as TECOPHILIC® HP-60D-60 and mixtures thereof, commercially available from the Lubrizol Corporation in Wickliffe, Ohio). Other suitable materials include other hydrophilic polymers such as hydrophilic polyether block amide (e.g., PEBAX® MV1074 and MH1657, commercially available from Arkema headquartered in King of Prussia, Pa.), hydrophilic nylons, hydrophilic polyesters, block co-polymers with built-in hydrophilic blocks, polymers including ionic conductors, polymers including electrical conductors, metallic or nanoparticle filled polymers, and the like. Suitable hydrophilic polymers may exhibit between 20% to 120% water uptake (or % water absorption) due to their hydrophilic nature or compounding. In at least some embodiments, first and third layers 350/354 may include a hydratable polymer that is blended with a non-hydratable polymer such as a non-hydratable polyether block amide (e.g., PEBAX® 7033 and 7233, commercially available from Arkema) and/or styrenic block copolymers such as styrene-isoprene-styrene. These are just examples.

The intermediate layer 352 may include an electrically non-conductive polymer such as a non-hydrophilic polyurethane, homopolymeric and copolymeric polyurethanes (e.g., NeoRez R-967, commercially available from NeoResins, Inc. in Wilmington, Mass.; and/or TECOFLEX® SG-85A and/or TECOFLEX SG-60D, commercially available from Lubrizol Corp. in Wickliffe, Ohio), polyether block amide, nylon, polyester or block-copolymer. Other suitable materials include any of a range of electrically non-conductive polymers. These are just examples.

The materials of the inner layer 350 and the intermediate layer 352 may be selected to have good bonding characteristics between the two layers. Similarly, the material of the outer layer layer 354 may be selected to have good bonding characteristics with the inner and intermediate layers 350, 352. For example, a balloon 316 may be formed from an inner layer 350 made from a hydrophilic polyether block amide and an intermediate layer 352 made from a regular or non-hydrophilic polyether block amide. In some instances, the material of the outer layer 354 may be the same as the material of the inner layer 350, although this is not required. In other embodiments, a suitable tie layer (not illustrated) may be provided between adjacent layers. These are just examples.

Further, the virtual electrodes 318 may be generally hydrophilic portions of balloon 316 and accordingly may absorb fluid (e.g., the conductive fluid) resulting in a conductive region of the balloon 316. Energy exposed to the conductive fluid can be conducted to virtual electrodes 318 such that virtual electrodes 318 are capable of ablating tissue. Therefore, during operations, an area between the target treatment region and the virtual electrodes 318 forms a tissue/catheter interface and thus an ablation site.

The catheter shaft 310 may include multiple lumens therein, such as guidewire lumen 370 and fluid inlet lumen 372. It is further contemplated, that while not explicitly shown, a separate fluid outlet lumen in communication with fluid outlet 312 may also be provided. The lumens connecting the fluid inlet 320 and the fluid outlet 312 can be connected to a known system that supplies new fluid and collects an evacuated fluid to circulate a suitable conductive fluid through the balloon 316. Other configurations may be contemplated as well and may be well known to someone in the art. In some embodiments, the above noted lumens may be omitted.

The ablation electrode 314 may extend along an outer surface of the catheter shaft 310 or may be embedded within the catheter shaft 310, adjacent a distal end region 322 of the catheter shaft 310. The ablation electrode 314 may be a wire filament based electrode made from either a single material or a combination of materials, such as platinum, gold, stainless steel, cobalt alloys, or other non-oxidizing materials. In some embodiments, such materials, when applied, may also be clad with copper. In some instances, titanium, tantalum, or tungsten may be applied as well. The ablation electrode 314 may extend substantially along the longitudinal length 311 of the balloon 316 or may extend only as far as the distal edge of the most distal virtual electrode 318. In general, the ablation electrode 314 may be helically wrapped around the catheter shaft 310. While the ablation electrode 314 is illustrated as having adjacent windings contacting one another, some embodiments may include windings that are spaced at a distance from each another. Alternatively, the ablation electrode 314 may have a linear or other suitable configuration. In certain cases, the ablation electrode 314 may be bonded to the catheter shaft 310. Moreover, the ablation electrode 314 and virtual electrodes 318 may be arranged such that the ablation electrode 314 extends directly under the virtual electrodes 318.

In some embodiments, the ablation electrode 314 may be ribbon shaped or may be a tubular member structured around the catheter shaft 310. In other embodiments, a plurality of ablation electrodes 314 may be employed, where each of them may be fixed to the catheter shaft 310, while sharing a common conductivity connection. In such embodiments that include more than one ablation electrode 314, each of the ablation electrodes 314 may be separately controllable as well. Further, the balloon 316 may be partitioned into more than one chamber and each chamber may include one or more such electrodes. Moreover, the ablation electrode 314 may be selected to provide a particular level of flexibility to the balloon to enhance the maneuverability of the device 300. Many other variations may be contemplated and applied for the ablation electrode 314, which may be known to someone in the art.

Extending over an outer surface of the catheter shaft 310 and the balloon 316 may be at least one optical fiber 302, which may be similar in form and function to the optical fiber 202 described in conjunction with FIG. 2. In the depicted embodiment, two such optical fibers 302 can be seen to extend linearly along the longitudinal length 311 of the balloon 316. The optical fibers 302 may include one or more FBG sensors 308, similar to FBG sensors 206, and those may be collectively termed as FBG sensors 308, hereinafter. It is contemplated that fewer or greater number of the optical fibers 302 may be employed as well. It is further contemplated that each optical fiber 302 may include more than one FBG sensor 308. In addition, configurations apart from a linear extension of the optical fiber 302 may be envisioned and applied by someone in the art. Furthermore, in some embodiments, the optical fiber 302 may be disposed either entirely or partially within a lumen, such as, but not limited to lumen 370, 372 (shown in FIG. 3B) of the catheter shaft 310. A proximal end (not shown) of the optical fiber 302 may extend to a read-out mechanism, such as read-out mechanism 204 (see FIG. 2), which may transmit light and measure the wavelength reflected by a FBG temperature sensor 308.

Thus, the FBG temperature sensors 308 may be positioned adjacent to the ablation electrode 314. More specifically, the optical fiber 302 extending over the balloon 316 enables the FBG sensors 308 formed within the optical fiber 302 to be positioned over the balloon 316 in such a way that they are adjacent to, in contact with, or meet the virtual electrodes 318. Such a configuration of the optical fiber 302 may enable a more accurate monitoring of temperature at an interface where the tissue meets the virtual electrode 318 during an ablation procedure. This interface may be referred to as a tissue catheter interface.

When performing an illustrative ablation procedure, an operator may steer and maneuver the balloon 316, positioned distally to the catheter shaft 310, towards a target site, positioning it adjacent to a target treatment region. A contact between the balloon 316 and the corresponding target treatment region may be enabled once the positioned balloon 316 is expanded to contact the lumen wall adjacent the target treatment region. Such an expansion of the balloon 316 may push the FBG sensors 308 into contact with the lumen wall as well, thus enabling the FBG sensors 308 to form a tissue catheter interface by lying between the tissue and the balloon 316. Thereafter, through suitable well known actuation mechanisms, an operator may power the renal nerve modulation device 300, to transfer an amount of energy to the ablation electrode 314 to perform ablation. The ablation electrode 314, being in contact with the lumen wall through the virtual electrodes 318, may ablate the target treatment region, while the optical fiber 302 disposed over the balloon 316 may monitor the temperature at the interface where the ablation procedure is being carried out. Such monitoring may allow an operator to control the ablation site's temperature more accurately.

As noted above, such temperature monitoring may be carried out through an optical read-out mechanism, optically coupled to the optical fiber 302, which transmits light into the optical fiber 302 and detects and receives light reflected from the one or more FBG sensors 308 placed within the optical fiber 302. Upon receiving the reflected light, the optical read-out mechanism encodes local or in situ temperature information determined at the tissue catheter interface(s) through the each of the one or more FBG sensors 308 deployed at that interface.

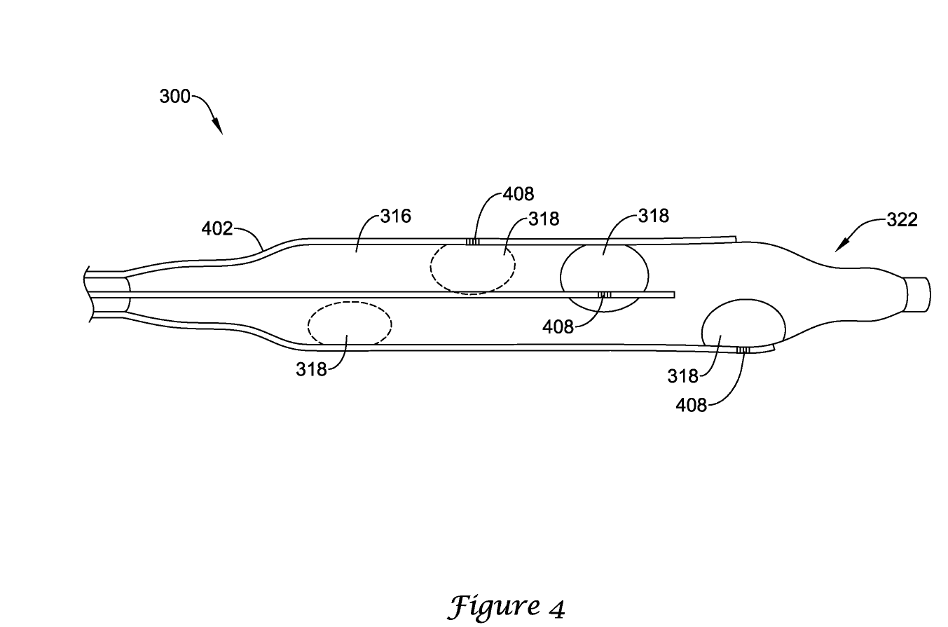

FIG. 4 depicts a side view of the illustrative embodiment of the device 300, illustrated in FIG. 3A, having a number of optical fibers 402. The optical fibers 402 may be circumferentially spaced about the catheter shaft 310 and may be similar in form and function to the optical fiber 202 discussed in connection with FIG. 2. While the optical fibers 402 are generally illustrated as uniformly spaced, it is contemplated that the optical fibers 402 may be spaced at any distance from one another, evenly or unevenly, as desired. Further, the optical fibers 402 shown here may be linearly disposed over the balloon 316 and may extend from the proximal end of the renal nerve modulation device 300 in a direction generally parallel to a longitudinal axis of the catheter shaft 310 and/or balloon 316, although this is not required. The optical fiber 402 may include corresponding FBG sensors 408. Further, while three of optical fibers 402 are illustrated, it is contemplated that greater than or fewer than three optical fibers 402 may be used. It is contemplated that in some instances, the length of the optical fibers 402 may vary, although this is not required. In some embodiments, the number of optical fibers 402 may correspond to the number of virtual electrodes 318, although this is not required. As noted above, the fiber Bragg grating sensor 408 may be positioned adjacent a corresponding virtual electrode 318 that functions to carry out an ablation procedure at/around a target treatment region. Other configurations, variations in sizing and positioning of the optical fibers 402, may be contemplated as well and, accordingly, the configurations disclosed in the application need not be seen as limiting in any way.

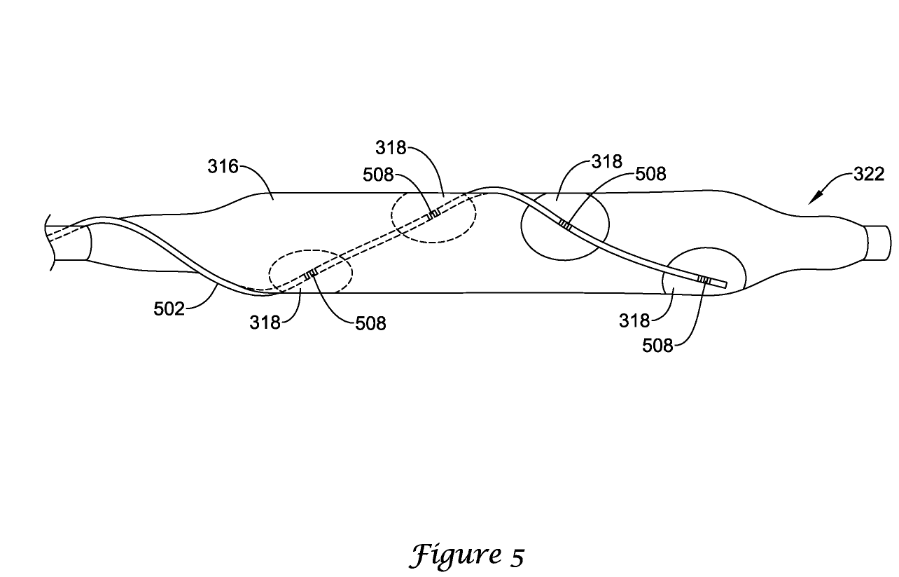

FIG. 5 illustrates a distal portion of an illustrative nerve modulation device including an optical fiber 502. The structure of the distal end region 322, balloon 316, and virtual electrodes 318 may be similar in form and function to those described above with respect to nerve modulation system 300. In the illustrative embodiment, the optical fiber 502 may initially extend from a proximal end of the renal nerve modulation device. While the optical fiber 502 may extend along the catheter shaft in a generally linear manner, towards the distal end the optical fiber 502 may start deflecting to form the helical configuration. In effect, the optical fiber 502 first extends approximately parallel to a longitudinal axis of the elongate shaft and winds into a helical formation towards the distal end region 322. The distal end region of the optical fiber 502 may be wound around the balloon 316 such that a portion of the optical fiber 502 is adjacent to one or more virtual electrodes 318. In some instances, a portion of the optical fiber 502 may be positioned adjacent to each of the virtual electrodes 318, although this is not required. It is contemplated that the optical fiber 502 may include one or more FBG sensors 508 positioned along its length. In some instances, one or more FBG sensors 508 may be spaced such that an individual sensor 508 is positioned adjacent to one or more virtual electrodes 318, although this is not required. As noted above, multiple FBG sensors 508 may be manufactured on a single optical fiber 502 such that each FBG sensor 508 has a unique reflection wavelength. Such wavelength division multiplexing makes it possible to differentiate between reflection signals from a plurality of FBG sensors 508 on a single optical fiber 502. As such, the temperature and/or other conditions may be monitored adjacent to one or more virtual electrodes 318 with a single optical fiber.

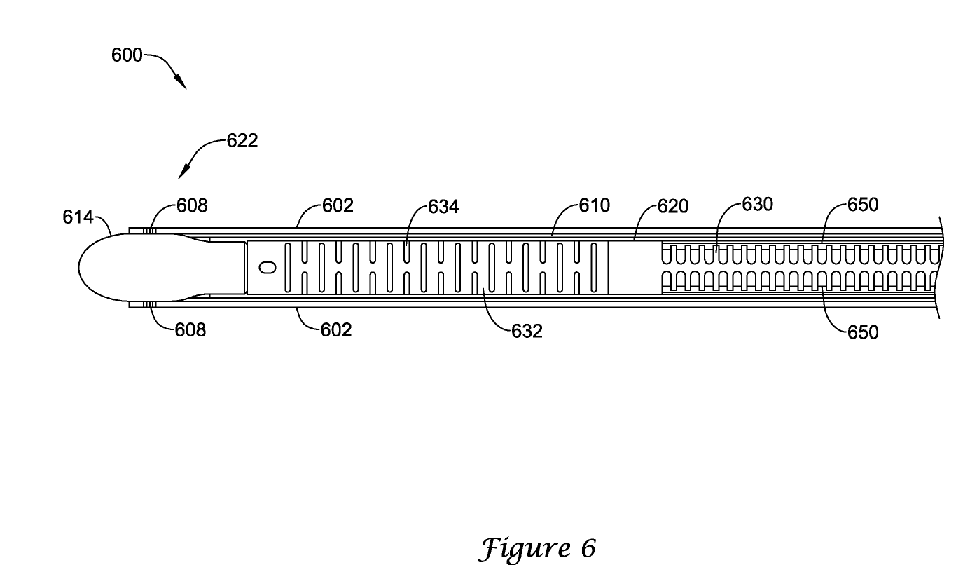

FIG. 6 illustrates a distal portion of another illustrative nerve modulation device 600 including an optical fiber 602. The device 600 may include a catheter shaft 610 and an electrode 614 positioned adjacent to the distal end region 622 of the catheter shaft 610. The electrode 614 may be formed at or otherwise form a distal tip of catheter shaft 610. In general, electrode 614 may be configured to ablate target tissue at or near a body lumen. For example, electrode 614 may be used to ablate a renal nerve adjacent to a renal artery. The electrode 614 may vary and may include a number of structures such as a plurality of wires (e.g., two wires) that connect with electrode wire and, ultimately, a control and power element.

In order to more specifically place or steer catheter 610 to a position adjacent to the intended target, catheter 610 may be configured to be deflectable. More specifically, the catheter shaft 610 may include a tubular member 620 that includes a flex body 630 that can be selectively bent. This allows a user to orient the electrode 614 in a desirable position/direction within a body lumen. To effect deflection, one or more pull wires or actuation members 650 may be coupled to the flex body 630. This may allow a user to actuate (e.g., “pull”) one or more of the pull wires causing the flex body 630 to deflect, and thus, catheter shaft 610 (e.g., electrode 614). Here, more specifically, the catheter shaft 610 bends, as desired, to allow an operator to place the electrode 614 adjacent a vessel wall. The actuation members may be sufficiently stiff to provide a pushing/pulling force on the flex body 630, for example, to straighten or deflect the flex body 630. Other embodiments of actuation mechanisms, which create or effect a catheter deflection may be contemplated and applied by those skilled in the art.

To further aid in properly orienting the catheter shaft 610 within a body lumen a flex tube 632 may be coupled to the flex body 630, for example, at a distal end of the flex body 630. The flex tube 632 may have a plurality of slots 634 formed therein to form a substantially flexible structure. In general, the flex tube 632 may be configured to be flexible so that distal portion of the catheter shaft 610 (e.g. adjacent to electrode 614) can bend upon encountering the wall of a lumen body. Accordingly, the flex tube 632 can bend when/if a distal end region 622 of the device 600 engages the wall of the lumen body during operations, atraumatically allowing the electrode 614 to follow along the wall of the lumen body. In some embodiments, the flex body 630 and flex tube 632 are two distinct structures that are attached to each another, while in certain embodiments the two may be made as a unitary structure.

Accordingly, varying levels of flexibility may be achieved along the length of the device 600, and, more particularly, the catheter shaft 610, through features such as these. Further, the mechanisms to operate and engage such features during applications may be known to someone in the art. A variety of other configurations and arrangements can also be utilized to accomplish a similar task without departing from the scope and spirit of the present disclosure.

One or more optical fibers 602, including one or more FBG sensors 608 similar to FBG sensors 206, can extend along the longitudinal length of the device 600 to substantially terminate adjacent the distal end region 622. The optical fibers 602 may be similar in form and function to the optical fiber 202 discussed in connection with FIG. 2. In some instances, the optical fibers 602 may extend generally parallel to a longitudinal axis of the catheter shaft 610, although this is not required. It is contemplated that the optical fibers 602 may be disposed along the catheter shaft 610 in any manner desired. For example, some portions of the optical fibers 602 may be helically wound. The FBG sensors 608 may be disposed adjacent the electrode 614 and are, more particularly, configured to be disposed between the electrode 614 and a corresponding vessel wall. Such a configuration of the optical fiber 602 and/or FBG sensors 608 may enable a more accurate monitoring of temperature at an interface where the tissue meets the electrode 614 during an ablation procedure. This interface may be referred to as a tissue catheter interface. In some embodiments, the number of optical fibers 602 may vary in fewer or greater numbers than the two optical fibers 602, exemplarily illustrated. Moreover, during applications, as only one portion of the electrode 614 may generally contact the vessel wall, only a single optical fiber 602 may be used at a time, although this is not required. It is further contemplated that each optical fiber 602 may include more than one FBG sensors 608 spaced along the length of the optical fiber 602, as desired.

As noted above, such temperatures monitoring may be carried out through an optical read-out mechanism, optically coupled to the optical fiber 602, which transmits light into the optical fiber 602 and detects and receives light reflected from the one or more FBG sensors 608 placed within the optical fiber 602. Upon receiving the reflected light, the optical read-out mechanism encodes local or in situ temperature information determined at the tissue catheter interface(s) through each of the one or more FBG sensors 608 deployed at that interface.

In some embodiments, the catheter shaft 610 may also include a number of additional features commonly associated with medical devices. For example, the catheter shaft 610 may include radiopaque markers or bands, additional or alternative catheter shaft constructions (e.g., having lumens, reinforcements, balloons, or other catheter structures), a proximal hub and strain relief, and the like.

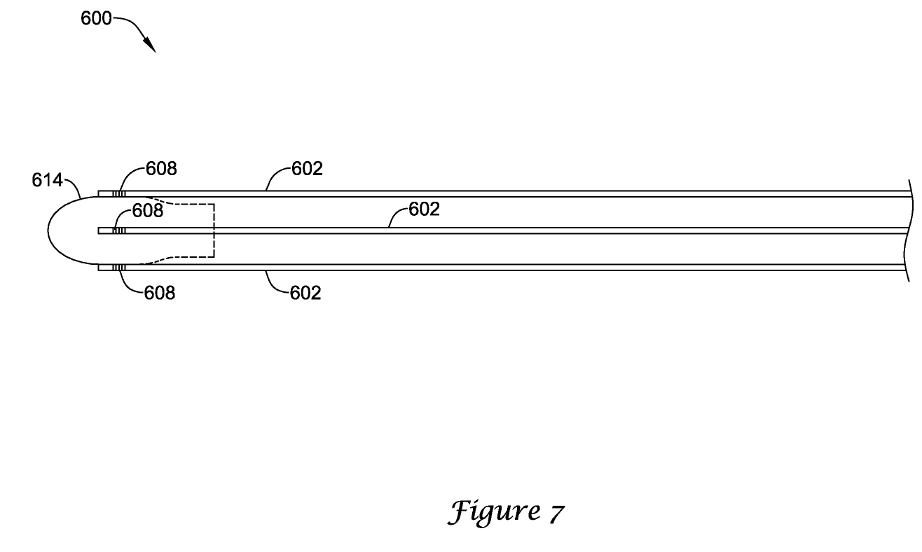

FIG. 7 depicts a side view of the device 600, including multiple optical fibers 602 disposed over an outer surface of the electrode 614 and the catheter shaft 610. The optical fibers 602 may be circumferentially spaced about the catheter shaft 610. While the optical fibers 602 are generally illustrated as uniformly spaced, it is contemplated that the optical fibers 602 may be spaced at any distance from one another, evenly or unevenly, as desired. All such optical fibers 602 disposed over the outer surface of the electrode 614 can have the FBG sensors 608 embedded therein, and may be configured to be flexible as well. Accordingly, the optical fibers 602 may be suited to accommodate flexible movement when maneuvering within a body lumen. Moreover, as noted above, the optical fibers 602 may extend along the length of the device 600 to at least substantially cover the longitudinal length of the device 600.

In all the embodiments described so far, materials used to manufacture the ablation devices described herein along with other components that may interact with portions of the human body, may include a rigid and/or a flexible material either in combination or alone. Accordingly, exemplary materials may also include metals, polymers, alloys, composite, or the like, either in combination or alone.

Those skilled in the art will appreciate that the different embodiments of the devices 300 and 600 described here, their modes of operation, etc., are merely representative of the environment in which the present disclosure operates. Accordingly, a variety of alternatively shaped collaborating components may also be used as a substitute for the purpose of monitoring an ablation temperature at a desired target site, thus, not limiting the scope of the present disclosure. Further, the optical fibers 202, 302, 402, 502, 602 may be adequately stretchable, extendable, and retractable, allowing for its flexible deployment during maneuvering and positioning. More particularly, the configurations of the optical fibers 202, 302, 402, 502, 602 described here may be applicable for other medical applications as well, and accordingly, a variety of other medical devices may be used in combination. Those medical devices may include biopsy forceps, scissors, lithotripters, dilators, other cautery tools, and all devices, that may require monitoring of temperature at a remote or hard to reach place.

Further, while the optical fibers 202, 302, 402, 502, 602, and 702, are generally described along an outer surface of a medical device, a variety of other dispositional configurations and arrangements may also be contemplated and conceived. For example, the optical fibers 202, 302, 402, 502, 602 may extend within a lumen of the device or a wall of the device along at least a portion of the length of the device, Embodiments of the present disclosure are thus suitably applicable to both medical and/or non-medical environments. Further, certain aspects of the aforementioned embodiments may be selectively used in collaboration, or removed, during practice, without departing from the scope of the disclosed embodiments.

Those skilled in the art will recognize that the present disclosed subject matter may be manifested in a variety of forms other than the specific embodiments described and contemplated herein. Accordingly, departure in form and detail may be made without departing from the scope and spirit of the present disclosure as described in the appended claims.

What is claimed is:

1. A medical system for performing ablation, the system comprising:
an elongate shaft having a proximal end region and a distal end region;
an ablation electrode disposed adjacent the distal end region of the elongate shaft;
a first optical fiber having a proximal end and a distal end, the first optical fiber extending along an outer surface of the elongate shaft and including one or more fiber Bragg grating (FBG) sensors;
an optical read-out mechanism optically coupled to the first optical fiber, the optical read-out mechanism configured to transmit light into the first optical fiber and detect light reflected from the one or more FBG sensors, the detected light reflected from the one or more FBG sensors encoding local temperatures at each of the one or more FBG sensors; and
wherein at least one of the one or more FBG sensors is positioned adjacent to the ablation electrode.

2. The medical system of claim 1, wherein the first optical fiber extends approximately parallel to a longitudinal axis of the elongate shaft.

3. The medical system of claim 1, wherein the distal end of the first optical fiber is helically wound around the distal end region of the elongate shaft.

4. The medical system of claim 1, further comprising one or more additional optical fibers extending along the outer surface of the elongate shaft and including one or more additional fiber Bragg grating (FBG) sensors.

5. The medical system of claim 4, wherein the one or more additional optical fibers are circumferentially spaced about the elongate shaft.

6. The medical system of claim 1, wherein the one or more FBG sensors are spaced along a length of the first optical fiber.

7. A medical device for performing ablation, the medical device comprising:
an elongate shaft having a distal end region;
a balloon coupled to the distal end region, the balloon including a conductive layer and a non-conductive layer;
an electrode disposed within the balloon;
a virtual electrode defined at a position along the balloon that is free of the non-conductive layer;
a first optical fiber having a proximal end and a distal end, the first optical fiber extending along an outer surface of the elongate shaft and including one or more fiber Bragg grating (FBG) temperature sensors; and
an optical read-out mechanism optically coupled to the first optical fiber, the optical read-out mechanism configured to transmit light into the first optical fiber and detect light reflected from the one or more FBG temperature sensors, the detected light reflected from the one or more FBG temperature sensors encoding local temperatures at each of the one or more FBG temperature sensors.

8. The medical device of claim 7, wherein at least one of the one or more FBG temperatures sensors is positioned adjacent to the virtual electrode.

9. The medical device of claim 7, wherein the one or more FBG temperature sensors are spaced along a length of the first optical fiber.

10. The medical device of claim 7, further comprising one or more additional virtual electrodes.

11. The medical device of claim 10, wherein at least one of the one or more FBG temperature sensors is positioned adjacent to each virtual electrode.

12. The medical device of claim 11, wherein the distal end of the first optical fiber is helically wound around the balloon such that at least one of the one or more FBG temperature sensors is positioned adjacent to each virtual electrode.

13. The medical device of claim 7, further comprising one or more additional optical fibers extending along the outer surface of the elongate shaft and including one or more additional fiber Bragg grating (FBG) temperature sensors.

14. A deflectable medical device, comprising:
a catheter shaft having a distal end region;
an ablation electrode disposed adjacent to the distal end region of the catheter shaft;
a deflection mechanism coupled to the catheter shaft, the deflection mechanism including a deflection body and a pull wire coupled to the deflection body;
a flex member disposed adjacent to the deflection mechanism;
a first optical fiber having a proximal end and a distal end, the first optical fiber extending along an outer surface of the catheter shaft and including one or more fiber Bragg grating (FBG) temperature sensors; and
an optical read-out mechanism optically coupled to the first optical fiber, the optical read-out mechanism configured to transmit light into the first optical fiber and detect light reflected from the one or more FBG temperature sensors, the detected light reflected from the one or more FBG temperature sensors encoding local temperatures at each of the one or more FBG temperature sensors.

15. The deflectable medical device of claim 14, wherein at least one of the one or more FBG temperatures sensors is positioned adjacent to the ablation electrode.

16. The deflectable medical device of claim 14, wherein the first optical fiber extends approximately parallel to a longitudinal axis of the catheter shaft.

17. The deflectable medical device of claim 14, wherein the distal end of the first optical fiber is helically wound around the distal end region of the catheter shaft.

18. The deflectable medical device of claim 14, further comprising one or more additional optical fibers extending along the outer surface of the catheter shaft and including one or more additional fiber Bragg grating (FBG) temperature sensors.

19. The deflectable medical device of claim 18, wherein the first optical fiber and the one or more additional optical fibers are circumferentially spaced about the catheter shaft.

20. The deflectable medical device of claim 14, wherein the one or more FBG temperature sensors are spaced along a length of the first optical fiber.
